(19) United States
(12) Patent Application Publication
(10) Pub. No.: US 2006/0263238 A1
(43) Pub. Date: Nov. 23, 2006

(54) Decontamination of biological microbes using quaternary ammonium surfactants at elevated temperatures

(76) Inventor: Tony L. Buhr, King George, VA (US)

Correspondence Address: Naval Surface Warfare Center;Office of Counsel (Patents) (Code XDC1), 17320 Dahlgren Road, Dahlgren, VA, 22448-5100, US

(21) Appl. No.: 11/240,780

(22) Filed: Sep. 6, 2005

Related U.S. Application Data
(60) Provisional application No. 60/683,555, filed on May 19, 2005.

(51) Int. Cl. A61L 2/18

(52) U.S. Cl. 422/28

(57) ABSTRACT

A large area decontaminating method for biological spore populations uses the application of a quaternary ammonium surfactant in combination with an elevated temperature to decontaminate the spores.

### STATEMENT REGARDING FEDERALLY SPONSORED RESEARCH OR DEVELOPMENT

The invention described herein may be manufactured and used by or for the government of the United States of America for governmental purposes without the payment of any royalties thereon or therefore.

### FIELD OF THE INVENTION

The present invention provides a method for large-scale decontamination of biological microbes using quaternary ammonium surfactants at elevated temperatures.

### BACKGROUND

There are numerous strategies for biological decontamination. These include both general and specific decontamination strategies.—General decontamination strategies include using strong acids, strong bases, strong oxidizers such as Clorox (hypochlorite), or gases such as methyl bromide. The problem with these strategies is that they are environmentally unfriendly, highly toxic, and/or corrosive, which limits their applications. When used in decontamination processes, e.g., processes generally used over large areas of coverage, strong chemicals become problematic as environmentally toxic and hazardous to people. In particular, these options could not be used to decontaminate in situations that necessitated contact of the chemicals with human skin. Specific decontamination strategies would include utilizing enzymes or developing biologically based solutions. The difficulty with specific decontaminants are limited efficacy, especially over a wide temperature range or a short time frame, high costs, limited availability, short shelf lives, and problems related to the decontaminant being readily inactivated.

There is a need in the art to provide an effective and safe methodology for large-scale decontamination. The present invention addresses this and other needs.

### SUMMARY

The present invention includes a method for large-scale decontamination of biological microbes, consisting essentially of the steps of applying a quaternary ammonium surfactant to the contaminating biological microbes and applying an elevated temperature in combination with the applied quaternary ammonium surfactant effective for large-scale decontamination of the biological microbes. Preferred quaternary ammonium surfactants include hexadecyltrimethylammonium chloride, dicocodimethylammonium chloride, alkyldimethylbenzylammonium chloride and combinations thereof. The present invention also includes a large-scale decontaminated microbe product produced by the above-described method.

The present invention also includes a method for large-scale decontamination of biological microbes, comprising the steps of washing a biological microbe contaminant from a decontamination site into a waste water product, applying an elevated temperature to the contaminated waste water product and applying a quaternary ammonium surfactant in combination with the elevated temperature effective for decontamination of the biological microbes within the waste water product.

The present invention provides solutions to decontaminate biological organisms including bacterial endospores, particularly spores produced by Bacillus species. Decontamination (decon) is the process of killing, removing and/or inhibiting (neutralizing) any harmful agents including biological organisms and/or chemicals.

### BRIEF DESCRIPTION OF THE DRAWINGS

FIG. 1 is a graph showing decontamination of B. globigii spores with 1% hexadecyltrimethylammonium chloride at 25° C. and 70° C.;

FIG. 2 is a graph showing decontamination of B. globigii spores with 1% dicocodimethylammonium chloride at 25° C. and 70° C.; and,

FIG. 3 is a graph showing decontamination of B. globigii spores with 1% alkyldimethylbenzylammonium chloride at 25° C. and 70° C.

### DETAILED DESCRIPTION

The present invention provides a method for decontaminating biological spores using the application of a quaternary ammonium surfactant (also referred to herein as quaternary ammonium compound) at an elevated temperature. The method of the present invention includes large-scale decontamination of biological microbes by applying the elevated temperature to large-scale biological spores contamination, and within that temperature environment, applying a quaternary ammonium surfactant compound onto the contaminant microbes in a manner for effective for large-scale decontamination of the biological microbes. The application of the present invention to the microbe populations also may preferably include an acidic environment. The method of the present invention decontaminates the spores with minimal environmental or health risks while being effective for decontaminating biological microbes, such as endospores within the Bacillus endospores family, including B. globigii spores. This allows effective and rapid decontamination of large areas permitting use of these large area, such as for military or civilian operations.

The quaternary ammonium surfactant of the present invention has the general formula of:

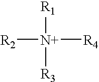

with R₁, R₂, R₃, and R₄ independently, being a substituted or unsubstituted alkyl or alkenyl group containing from about 1 to about 25 carbon atoms, and/or substituted or unsubstituted aromatic groups. Preferably, the quaternary ammonium surfactant of the present invention includes hexadecyltrimethylammonium chloride, dicocodimethylammonium chloride, alkyldimethylbenzylammonium chloride and combinations thereof. Quaternary ammonium surfactants are commercially available, such as from Akzo Nobel of Arnhem, Netherlands. The type and amount of quaternary ammonium surfactant useful for decontamination of a particular spore population may be determined by those skilled in the art of decontamination in light of the disclosure herein. Relevant factors useful in determining the most appropriate conditions to be used include such items as the type of spore species, type of surface or area to be decontaminated, amount of contamination, environmental conditions of the cleanup, and other such criteria that are determinable by those skilled in the art of decontamination. The quaternary ammonium surfactants are preferably applied in an aqueous medium, such as water, diol, or other conveyor of the quaternary ammonium surfactant, with such conveyor determinable by one skilled in the decontamination art in light of the disclosure herein.

The quaternary ammonium surfactant is applied in a heated condition, such as a moderately heated condition, to facilitate decontamination. The moderately heated condition of the spores includes above-ambient temperatures that in combination with the quaternary ammonium effectively decontaminate the spore population. Representative temperatures of a heated condition include, for example, non-destructive temperatures for a given object to be decontaminated, such as temperatures below the boiling point of an aqueous constituent of the object to be decontaminated or any constituent part of the acidic environment, e.g., from about 100° C. or less, from about 25° C. to about 100° C., from about 50° C. to about 100° C., from about 65° C. to about 85° C., from about 75° C. to about 80° C., and the like. Preferred temperatures include, for example without limitation, heating the biological spore environment to a temperature of from about 25° C. to about 70° C., or a more specific temperature of about 70° C. Methods of heating may include imparting heat into the immediate environment of the spore population including, for example, use of exothermic chemical reaction, use of external heat source such as heating elements, combinations of these methods, and the like.

Methodologies of the decontamination of the spores include the spores being subjected to the quaternary ammonium surfactant in solution and, simultaneously or sequentially, exposed to a heated condition. Heat may be applied prior to, during or after the application of the quaternary ammonium surfactant and/or a conveyor. The quaternary ammonium surfactant and heated conditions may be varied by the type and amount of spore population, and by the object or environment to be decontaminated. In one preferred embodiment, the biological spores are collected from a contaminated area and placed in a container for application of quaternary ammonium surfactant and heat, such as in the form of a heating element. Alternatively, the application of preheated dilute quaternary ammonium surfactant may be used for decontamination on site.

In one application of the present invention, spores may be harvested and/or trapped by vacuums, filters, glues, etc. to collect and concentrate the spores and placed into a container or similar retaining device. Within the container, the spores are exposed to appropriate quaternary ammonium surfactant/heated conditions, which may be present when the spores were placed in the container or added after placement of the spores, using a heating element for an appropriate time period to kill and decontaminate the spores. Such decontamination devices may be small, inexpensive and readily transportable, having a resilient container, such as a glass-based, resilient plastic or stainless steel composition, and appropriate heating component for the quantity to be heated, range of temperature to heat, and duration of heating. In one embodiment, an exothermic chemical reaction may be used in place of the heating element for imparting heat to the quaternary ammonium surfactant environment to kill spores in contaminated areas. Numerous factors, determinable by artisans in the decontamination arts, affect the efficiency of the decontamination method of the present invention. Such factors include, for example, the selection of quaternary ammonium surfactant, concentration of the quaternary ammonium surfactant, uniformity of heat, variations in heat temperatures, time period of application, type of spore, etc., with the optimum decontamination conditions determinable by those skilled in the art through routine experimentation in light of the disclosure herein. Generally, selection of appropriate decontamination procedures includes a balance of the quaternary ammonium surfactant strength, heat conditions, time constraints (such as operational military timetables), environmental sensitivities to elevated heated conditions, and toxicity for a given spore hazard. Preferably decontamination occurs within about 60 minutes, such as 15 minutes, 30 minutes or 45 minutes.

In one preferred embodiment, an acidic pH is used to increase sporicidal activity. Application of the acidic environment includes any appropriate methodology for exposing the spores, such as by suspending, surrounding, encasing, inundating, engulfing, submerging, misting, or otherwise subjecting the spores to the acidic environment as to affect the spores thereby. Methodologies may include sprays, mists, liquids, solids and the like. Preferably application of the acidic environment comprises application of an acidic solution to the spores or to an environment to which the spores are introduced. Acidic environments include, for example, pH ranges of less than about 6.9, preferably from about 1.0 to less than about 6.9, more preferably from about 3.0 to about 6.0, and still more preferably from about 4.0 to about 6.0, and most preferably about 5.0. Acidic solutions may include carboxylic acids, organic acids, inorganic acids, and combinations thereof, including such acids as oxalic acid, acetic acid, phosphoric acid, hydrochloric acid, citric acid, sulfuric acid, nitric acids, and the like. In solution the acids may be present in amounts that allow the convenient and non-hazardous application onto the spores. Such amounts may include, for example, from about 1 mM to about 1,000 mM, from about 2 mM to about 500 mM, from about 10 mM to about 100 mM, from about 20 mM to about 50 mM, and the like. When solutions are applied to a spore population, preferably enough solution is applied to completely immerse the spores in the solution. Application of the acidic environment may include application of an acidic solution in a moderately heated condition. Most preferably, the acid solution is selected for specific locations and uses to minimize harmful effects, such as harmful effects on humans or equipment.

The large-scale decontaminated microbe population produced by the present invention overcomes safety issues, particularly health problems, for the effective use of decontamination methods. Additionally, the present invention addresses on-going use of contaminated equipment, such as military aircraft, ships and the like, after decontamination of this equipment. The use of large amounts of a quaternary ammonium surfactant in a moderately heated environment has been found effective for and is less likely to be a health risk or cause material damage compared to other decontaminant procedures such as hypochlorite (bleach) or methyl bromide.

Bacterial endospores are one of the most difficult biological agents to kill and decontaminate. Success with decontamination of bacterial spores is a strong benchmark for the solutions to be effective over many types and forms of microbes. The heated quaternary ammonium surfactant solutions are not exclusive to decontamination of Bacillus endospores and can be used for spores of other bacteria including Clostridium, or other forms of bacteria including vegetative bacteria and other microbes including other species of bacteria, viruses and fungi. The present invention is particularly applicable for decontamination of biological spores, particularly biological spores that comprise bacterial endospores. As used herein, the terms spores, biological spores, spore populations and similar terminology, refer to contaminant spores that create a hazard, threat, nuisance, etc. by their presence in an environment, on a surface, in food, etc. Typical spores decontaminated by the present invention include, for example, endospores, such as those belonging to the genus Bacillus, Clostridium, and the like. Representative endospore populations include Bacillus and Clostridium species. Some examples are Bacillus subtilis, Bacillus anthracis and Bacillus globigii.

Effectiveness of the methodology of the present invention occurs with increased biological spore kills with the use of the quaternary ammonium surfactant/heated environment over the non-use of such conditions. Preferably, an effective kill is variable, depending on the original number of spores within a contamination, such as a 90% effectiveness (kill) against a concentration of 10³ spores/ml, and more preferably an effectiveness of 90% against a concentration of 10⁸ spores/ml, with a most preferred decontamination of from about six or more orders-of-magnitude reductions of live spores. Most preferably, the decontamination reduces the spore concentration to a level that renders the once hazardous contaminated area or surface no longer hazardous. Effective biological spore decontamination of these spores rids a contaminated space or object of the immediate hazard occasioned by the spore presence. Spores are killed when they are rendered harmless, i.e., no longer hazardous, to a living organism, particularly a human. Depending on the circumstances, spore decontamination may be desirable against spores that affect other mammals, animals or plants. Decontamination applications non-exclusively include decontamination of endospore-forming bacteria in military, medical, industry, agriculture, and household domains, particularly in the event of accidental contamination or terrorist attack. Representative localities that could benefit from the decontamination regimen of the present invention for reduction of spore populations include hospitals, veterinary clinics, farms, dairies, meat processing facilities, hide processing facilities, ships, buildings, houses, automobiles, and other like contaminated surfaces and/or areas.

Advantageously the present invention provides low cost, a likely great reduction in toxicity and improved materials compatibility compared to current highly reactive decontaminants, and reduced logistical problems for decontamination. Selection of quaternary ammonium concentrations, heat conditions, pH, and desired kill rates may be varied to treat specific contaminated environments or surfaces.

### Methodology

In the examples, below, three representative quaternary ammonium surfactants were tested for decontamination of Bacillus spores. The three exemplified quaternary ammonium surfactants are hexadecyltrimethylammonium chloride, dicocodimethylammonium chloride, and alkyldimethylbenzylammonium chloride. All three quaternary ammonium surfactants were dissolved in water and tested over a pH range. Testing occurred with and without applied heat. All experiments were performed with at least 3-4 replicates with many experiments reproduced numerous times.

Bacillus spores were suspended in the appropriate solvent at a concentration of 2 mg spores/ml (approximately 2×10⁸ spores/ml). Starting volume of 1.125 ml of spores at 2 mg/ml. 3 or 4 spore aliquots of 50 μl per aliquot were removed. These aliquots were serially diluted in 1×PBS and plated on LB or TSA agar plates. This is a control step that gave the spore titer or colony-forming units (CFUs) at Time=0 minutes (T0). The appropriate decontamination solution (2× concentration) was aliquoted into each of 3 or 4 Eppendorf tubes with 200 ul per tube. Then 200 ul of spores were added and mixed to give a final concentration of 1× decontamination solution and 1 mg spores/ml. The spores in decontamination solution were then mixed and incubated for specific time periods and temperatures. A typical incubation was in a room temperature (25° C.) water bath for 30 (or 60) minutes. There was no agitation during the incubation period in order to mimic real world situations. Aliquots of spores were removed after 30 (or 60) minutes. The aliquots (3 or 4 replicates) were serially diluted in 1×PBS and plated on LB or TSA agar plates. This gave a CFU at Time=30 minutes (T30), which is the titer of live spores after the decontamination test. After removing the aliquots at the 30-minute time point, test (or control) samples were then immediately incubated in a 70° C. water bath for another 30 minutes. The samples were then serially diluted and plated on to LB or TSA agar plates to determine the titer after the 70° C. incubation. The entire dilution series from 0.1 through 0.000001 dilutions were plated. An optional wash assay was employed to confirm that the surfactant was killing spores rather than behaving as a bacterostatic agent. The wash assay included centrifuging and washing spores after decontamination. 100 ul of spores (after decontamination) were added to 200 ul of water and centrifuged at 10,000×g for one minute at room temperature. The supernatant was removed and saved. 10% of the supernatant volume was plated and titered as a wash control, to determine if live spores were being removed in the supernatant. Spore pellets were suspended in 1 ml of water and then pelleted at 10,000×g for one minute at room temperature. The supernatant was removed and saved. Spore pellets were washed again with 1 ml of water. Then spore pellets were suspended, serially diluted and plated. This removed the surfactant prior to plating and confirmed results that were bactericidal rather than bacterostatic.

### EXAMPLE 1

### Spore Decontamination Using Hexadecyltrimethylammonium Chloride Suspended in Water with or Without Applied Heat

Hexadecyltrimethylammonium chloride was suspended in water. The hexadecyltrimethylammonium chloride was obtained from Akzo Nobel of Arnhem, the Netherlands, manufactured under the trade name Arquad 16-29. The hexadecyltrimethylammonium chloride was shipped as a 29% solution in water. Arquad 16-29 was either diluted in water without adjusting the pH, or samples were pH-adjusted using HCl or NaOH.

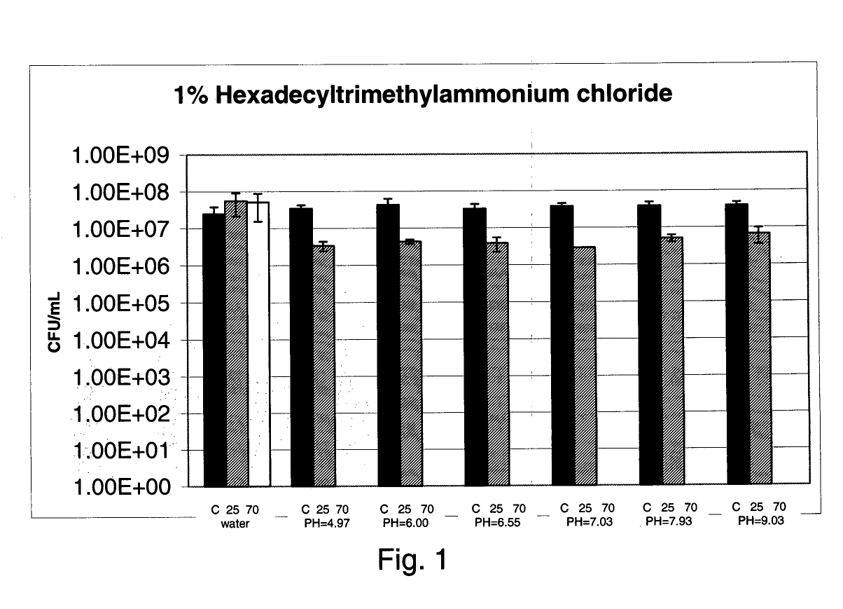

Referring to FIG. 1, decontamination of B. globigii spores is shown after spores were suspended in hexadecyltrimethylammonium chloride at a final concentration of 1%, and a spore concentration greater than 10⁷ spores per milliliter for 30 minutes at 25° C., or for 30 minutes at 70° C. The assay used to generate FIG. 1 was decontamination followed immediately by dilution and plating. As seen in FIG. 1, spore decontamination after 30 minutes at 25° C. (room temperature) resulted in an approximately 1 order-of-magnitude reduction of spores that appear independent of the pH (“25” designated bars in FIG. 1). There was a complete, greater than 7-orders-of-magnitude reduction of spores after 30 minutes at 70° C. (“70” designated bars in FIG. 1), and the results were apparently independent of pH. Thus moderate heat (70° C.) strongly increased the efficacy of decontamination compared to 25° C.

An optional wash assay was employed to verify the results (data not shown). The purpose of the wash assay was to remove the decontaminant (hexadecyltrimethylammonium chloride surfactant) prior to plating to reduce the likelihood that the surfactant was binding to spores and simply inhibiting growth, i.e., this assay confirmed there were no false positives in the previous assay. The wash assay showed over 4 orders-of-magnitude reduction of spore decontamination between pH5 and pH7 at 70° C. This assay indicated that less than 10 thousand spores were alive out of the original 10-100 million spores. Thus, results from both assays were very similar for acidic to neutral pH between pH5 and pH7. However, the wash assay indicated that decontamination at basic pH (pH8 and pH9) was between 1 and 3 orders-of-magnitude reduction at 70° C. Thus there is some discrepancy in efficacy at basic pH depending on the assay. Accordingly, hexadecyltrimethylammonium chloride may be suspended at acidic to neutral pH for the highest consistency and efficacy.

### EXAMPLE 2

### Spore Decontamination Using Dicocodimethylammonium Chloride Suspended in Water with or Without Applied Heat

Dicocodimethylammonium chloride was dissolved in water. Dicocodimethylammoniun chloride was obtained from Akzo Nobel of Arnhem, the Netherlands, manufactured under the trade name Arquad 2C-75. The Dicocodimethylammonium chloride was shipped as a 75% solution in water. Arquad 2C-75 was either diluted in water without adjusting the pH, or samples were pH-adjusted using HCl or NaOH.

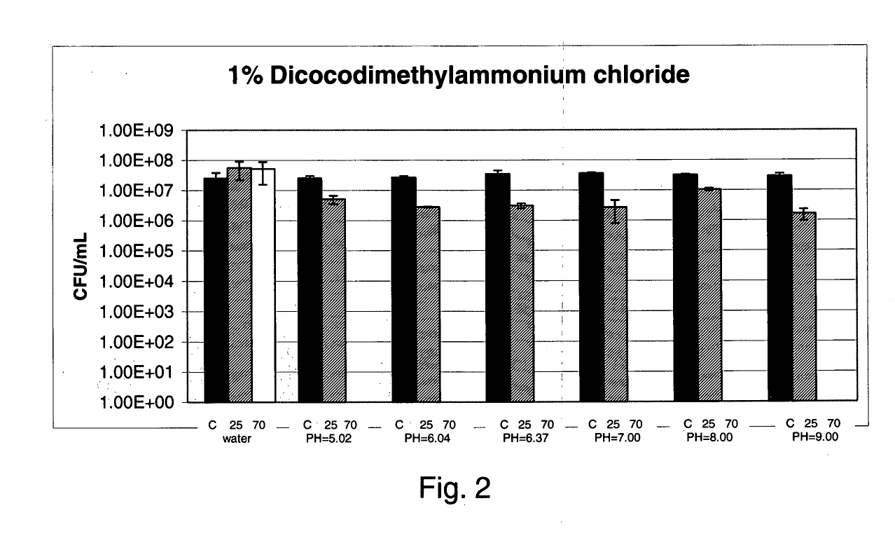

Referring to FIG. 2, decontamination of B. globigii spores is shown after spores were suspended in dicocodimethylammonium chloride at a final concentration of 1%, and a spore concentration greater than 10⁷ spores per milliliter for 30 minutes at 25° C., or for 30 minutes at 70° C. The assay used to generate FIG. 2 was decontamination followed immediately by dilution and plating. There was an approximately 1 order-of-magnitude reduction of spores after 30 minutes at 25° C. (room temperature), and the results were apparently independent of pH (“25” bars in FIG. 2). There was a complete, greater than 7 orders-of-magnitude reduction of spores after 30 minutes at 70° C., and the results were apparently independent of pH (“75” bars in FIG. 2). Thus moderate heat (70° C.) strongly increased the efficacy of decontamination compared to 25° C.

An optional wash assay was employed to verify the results (data not shown). The purpose of the wash assay was to remove the decontaminant (dicocodimethylammonium chloride surfactant) prior to plating to reduce the likelihood that the surfactant was binding to spores and simply inhibiting growth, i.e., this assay confirmed there were no false positives in the previous assay. The wash assay showed over 4 orders-of-magnitude reduction of spore decontamination between pH5 and pH7 at 70° C. This assay indicated that less than 10 thousand spores were alive out of the original 10-100 million spores. Thus, results from both assays were very similar for acidic to neutral pH between pH5 and pH7. However, the wash assay indicated that decontamination at basic pH (pH8 and pH9) was between 1 and 3 orders-of-magnitude reduction at 70° C. Thus there is some discrepancy in efficacy at basic pH depending on the assay. Accordingly, the dicocodimethylammonium chloride may be suspended at acidic to neutral pH for the highest consistency and efficacy.

### EXAMPLE 3

### Spore Decontamination Using Alkyldimethylbenzylammonium Chloride Suspended in Water with or Without Applied Heat

Alkyldimethylbenzylammonium chloride was dissolved in water. Mason Chemical Company manufactures the surfactant under the trade name Maquat MC1412. It is shipped as a 50% solution in water. Maquat MC1412 was either diluted in water without adjusting the pH, or samples were pH-adjusted using HCl or NaOH.

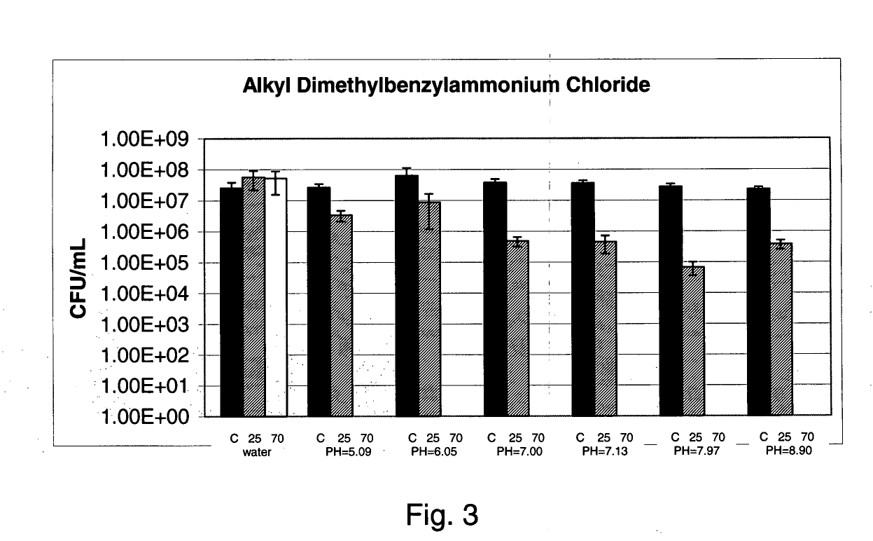

FIG. 3 shows decontamination of B. globigii spores after spores were suspended in alkyldimethylbenzylammonium chloride at a final concentration of 1%, and a spore concentration greater than 10⁷ spores per milliliter for 30 minutes at 25° C., or for 30 minutes at 70° C. The assay used to generate FIG. 3 was decontamination followed immediately by dilution and plating. There was an approximately 1 order-of-magnitude reduction of spores after 30 minutes at 25° C. (room temperature), and the results were apparently independent of pH (“25” bars in FIG. 3). There was a complete, greater than 7 orders-of-magnitude reduction of spores after 30 minutes at 70° C., and the results were apparently independent of pH (“75” bars in FIG. 3). Thus moderate heat (70° C.) strongly increased the efficacy of decontamination compared to 25° C. The optional wash assay was not tested for alkyldimethylammonium chloride.

The three quaternary ammonium compounds (hexadecyltrimethylammonium chloride, dicocodimethylammonium chloride, alkyldimethylbenzylammonium chloride) tested were each shown to have strong sporicidal (bacterial) activity in the presence of moderate applied heat. Each of the three surfactants tested were also tested in conjunction with ethanol as a solvent at ethanol concentrations ranging from 25% to 75% (data not shown). The sporicidal results using ethanol as a solvent was identical to the results using water as a solvent. Thus the surfactants could be suspended in ethanol for decontamination, and the alcohol should have no antagonistic affects on the surfactants.

### EXAMPLE 4

### Prophetic

Decontamination teams arrive at a contaminated area and set up an operations tent for decontamination. The teams conduct a large scale wash-down of individuals and equipment, and collect the used water (“gray water”) in holding containers. The gray water is heated and a quaternary ammonium compound is added. The holding containers remain heated with external heating elements, holding the gray water for one hour at 70° C. The gray water is then discarded. The holding containers are used again for decontaminating another batch of contaminated waste water.

### EXAMPLE 5

### Prophetic

Decontamination teams arrive at a contaminated area and set up an operations tent for decontamination. The teams conduct a large scale wash-down of individuals and equipment using a quaternary ammonium surfactant at a moderately elevated temperature, and collect the used wash solution (“gray washdown”) in holding containers. The gray washdown in the holding containers is heated by circulating the gray water through a radiator from an internal combustion engine, which is typically maintained at 90° C.-95° C. The average temperature of the gray washdown in the holding tank is maintained at 70° C. or higher for 30 minutes or longer by circulating the large volume of water through the radiator. The decontaminated gray washdown is then recycled or discarded. The holding containers are used again for decontaminating another batch of contaminated gray washdown.

Decontamination applications for the present invention include military, medical, industry, agriculture, household areas, such as from accidental contamination or in the event of terrorist attacks. The decontamination solutions are useful for reducing microbe populations in hospitals, veterinary clinics, farms, dairies, meat processing facilities, hide processing facilities, ships, buildings, houses, automobiles, and other various contaminated surface and/or area, including for example skin, hair and clothes. The efficacy against bacterial spores is a strong indicator that quaternary ammonium surfactants, preferably at acidic to neutral pH, plus moderate heat will be effective against a wide range of biological agents because bacterial spores are considered one of the most difficult to decontaminate. Effectiveness of the quaternary ammonium solution may include removal of many biological agents, including Bacillus endospores and other bacteria including Clostridium, or nonspore-forming bacteria such as vegetative bacteria and other microbes including fungi, viruses and possibly toxins. The quaternary ammonium surfactants are generally inexpensive, highly available and have a long shelf life.

The foregoing summary, description, and examples of the present invention are not intended to be limiting, but are only exemplary of the inventive features which are defined in the claims.

What is claimed is:

1. A method for large-scale decontamination of biological microbes, consisting essentially of the steps of:
applying a quaternary ammonium surfactant to the contaminating biological microbes; and,
applying an elevated temperature in combination with the applied quaternary ammonium surfactant effective for large-scale decontamination of the biological microbes.

2. The method of claim 1, further comprising the step of applying an acidic environment to large-scale biological spores contamination.

3. The method of claim 1, wherein the quaternary ammonium surfactant is selected from the group consisting of hexadecyltrimethylammonium chloride, dicocodimethylammonium chloride, alkyldimethylbenzylammonium chloride and combinations thereof.

4. The method of claim 3, wherein the quaternary ammonium surfactant comprises hexadecyltrimethylammonium chloride.

5. The method of claim 3, wherein the quaternary ammonium surfactant comprises dicocodimethylammonium chloride.

6. The method of claim 3, wherein the quaternary ammonium surfactant comprises alkyldimethylbenzylammonium chloride.

7. The method of claim 1, wherein the elevated temperature includes a moderately heated condition.

8. The method of claim 7, wherein the elevated temperature comprises a temperature of from about 25° C. to about 95° C.

9. The method of claim 8, wherein the elevated temperature comprises a temperature of from about 60° C. to about 80° C.

10. The method of claim 1, wherein the elevated temperature is produced by a heating event selected from the group consisting of exothermic chemical reaction, external heat source, and combinations thereof.

11. The method of claim 10, wherein the elevated temperature is produced by an external heat source.

12. The method of claim 10, wherein elevated temperature is produced by an exothermic chemical reaction.

13. The method of claim 2, wherein the step of applying an acidic environment comprises application of an acidic solution.

14. The method of claim 13, wherein the step of applying an acidic environment comprises a pH of from about 3.0 to about 6.0.

15. The method of claim 13, wherein the acidic solution is selected from the group consisting of organic acids, inorganic acids and combinations thereof.

16. The method of claim 2, wherein the acidic solution is selected from the group consisting of oxalic acid, acetic acid, phosphoric acid, hydrochloric acid, sulfuric acid, carboxylic acids, and combinations thereof.

17. The method of claim 1, wherein the biological microbes comprises endospores.

18. The method of claim 1, wherein the biological microbes comprises Bacillus endospores.

19. The large-scale decontaminated microbe product produced by the method of claim 1.

20. A method for large-scale decontamination of biological microbes, comprising the steps of:
washing a biological microbe contaminant from a decontamination site into a waste water product;
applying an elevated temperature to the contaminated waste water product; and,
applying a quaternary ammonium surfactant in combination with the elevated temperature effective for decontamination of the biological microbes within the waste water product.
